(19) United States
(12) Patent Application Publication
(10) Pub. No.: US 2011/0245839 A1
(43) Pub. Date: Oct. 6, 2011

(54) LOCKING SCREW DRIVER WITH INCREASED TORSIONAL STRENGTH

(75) Inventor: Jerry L. Lower, Bourbon, IN (US)

(73) Assignee: Zimmer, Inc., Warsaw, IN (US)

(21) Appl. No.: 12/752,507

(22) Filed: Apr. 1, 2010

(51) Int. Cl. A61B 17/56; B23P 11/00

(52) U.S. Cl. 606/104; 29/428

(57) ABSTRACT

An orthopedic tool for implanting a bone screw and a method of manufacturing the same. The orthopedic tool includes a head that is shaped to lock onto the bone screw and that has improved torsional strength.

### BACKGROUND

1. Field of the Invention

The present invention relates to an orthopedic tool. More particularly, the present invention relates to an orthopedic tool for implanting bone screws, and to a method of manufacturing the same.

2. Description of the Related Art

Orthopedic components, such as prosthetic joints and bone plates, may be secured to a patient's bone using bone screws. For example, a surgeon may position a bone plate to extend across a fracture line, and then the surgeon may secure the bone plate in place by inserting a plurality of bone screws through apertures in the bone plate and into the patient's bone.

To facilitate proper alignment of the bone screw within each aperture of the bone plate and to ease the insertion thereof, the surgeon may utilize a guide wire and a cannulated bone screw. First, the surgeon may insert the guide wire into the patient's bone at the point where the surgeon intends for the bone screw to be positioned. Then, the surgeon may slide the cannulated bone screw along the guide wire until reaching its intended position on the patient's bone.

With the cannulated bone screw in its intended position, the surgeon may engage a head of the bone screw with a driver. The driver may also be cannulated so that, like the bone screw, the driver may be guided to the intended position using the guide wire.

Bone screws and their corresponding drivers may be quite small in size. As a result, the components may have limited torsional strength. Hollowing out the bone screws and their corresponding drivers to create cannulated components that accommodate a guide wire may further limit the strength of the components.

### SUMMARY

The present invention provides an orthopedic tool for implanting a bone screw and a method of manufacturing the same. The orthopedic tool includes a head that is shaped to lock onto the bone screw and that has improved torsional strength.

According to an embodiment of the present invention, a method is provided for manufacturing an orthopedic tool. The method includes the steps of: providing a head shaped in a first configuration, the head having a longitudinal axis, a first end, a second end, and a plurality of sides that extend from the first end to the second end, the plurality of sides defining a non-circular cross section in a direction perpendicular to the longitudinal axis; applying torque to the head to shape the head into a second configuration that differs from the first configuration, the plurality of sides extending helically about the longitudinal axis in the second configuration; and coupling the head to a handle.

According to another embodiment of the present invention, a method is provided for manufacturing an orthopedic tool for use with a bone screw, the bone screw defining a socket with a non-circular cross section. The method includes the steps of: providing a head shaped in a first configuration, the head having a longitudinal axis, a first end, a second end, and a plurality of sides that extend from the first end to the second end, the plurality of sides defining a non-circular cross section in a direction perpendicular to the longitudinal axis; applying torque to the head to shape the head into a second configuration that differs from the first configuration, the head sized to be inserted and removed from the socket of the bone screw while shaped in the second configuration; and coupling the head to a handle.

According to yet another embodiment of the present invention, an orthopedic tool is provided for use with a bone screw, the bone screw defining a socket with a non-circular cross section. The orthopedic tool includes a handle and a head shaped in a second configuration and coupled to the handle, the head having a longitudinal axis, a first end, a second end, and a plurality of sides that extend from the first end to the second end, the plurality of sides defining a non-circular cross section in a direction perpendicular to the longitudinal axis. The head is manufactured by the steps of providing the head shaped in a first configuration that differs from the second configuration and applying torque to the head to shape the head into the second configuration, the head sized to be inserted and removed from the socket of the bone screw while shaped in the second configuration.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a perspective view of a bone screw having a socket;

FIG. 2 is a plan view of an exemplary orthopedic tool of the present invention, the orthopedic tool having a head shaped in a second configuration;

FIG. 3A is a perspective view of the head of FIG. 2 shaped in a first configuration;

FIG. 3B is a plan view of the head of FIG. 3A;

FIG. 3C is an elevational view of the head of FIG. 3A;

FIG. 4A is a perspective view of the head of FIG. 2 shaped in the second configuration;

FIG. 4B is a plan view of the head of FIG. 4A; and

FIG. 4C is an elevational view of the head of FIG. 4A.

Corresponding reference characters indicate corresponding parts throughout the several views. The exemplification set out herein illustrates an exemplary embodiment of the invention and such exemplifications are not to be construed as limiting the scope of the invention in any manner.

### DETAILED DESCRIPTION

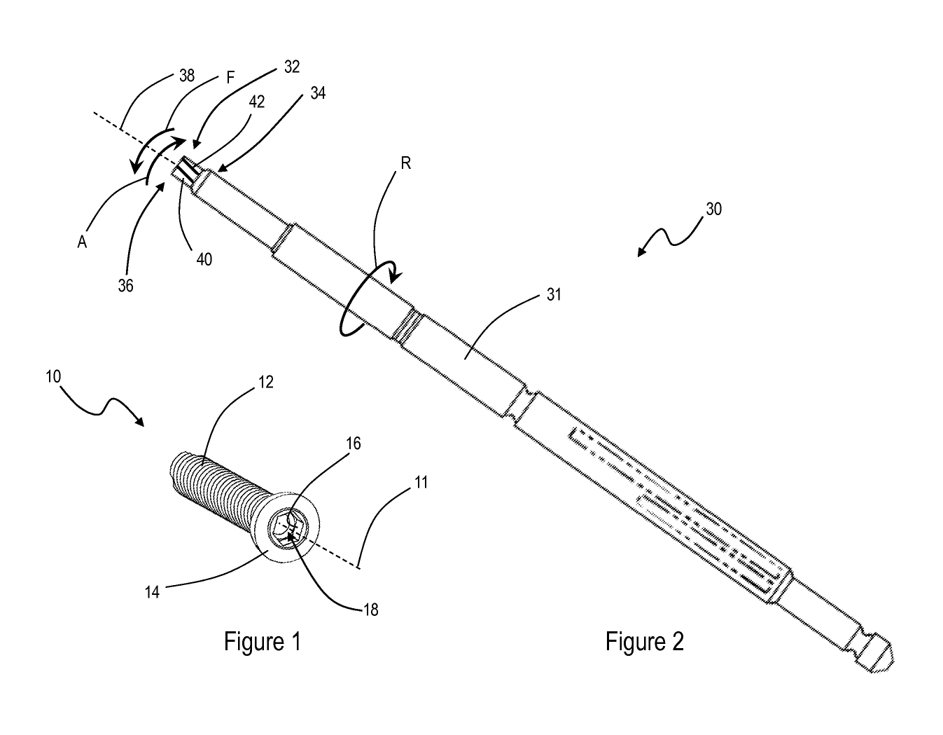

FIG. 1 depicts an exemplary bone screw 10. Bone screw 10 extends along longitudinal axis 11 and includes threaded shaft 12 and head 14. Head 14 of bone screw 10 includes walls 16 that define internal socket 18 having a non-circular cross section. According to an exemplary embodiment of the present invention, socket 18 is polygonal in shape. For example, walls 16 may define socket 18 that is hexagonal in shape, as shown in FIG. 1, triangular in shape, rectangular in shape, or octagonal in shape.

To facilitate alignment and insertion of bone screw 10 into a patient's bone, bone screw 10 may be cannulated. As shown in FIG. 1, bone screw 10 includes longitudinal bore 20 that extends entirely through threaded shaft 12 and head 14 of bone screw 10 along longitudinal axis 11. In use, a surgeon may pass a guide wire (not shown) through longitudinal bore 20 of bone screw 10 to guide positioning of bone screw 10.

Bone screw 10 is configured to mate with a corresponding orthopedic tool, such as driver 30 of FIG. 2. Driver 30 includes handle 31 and head 32 that is sized and shaped for receipt within socket 18 of bone screw 10. In the illustrated embodiment, head 32 of driver 30 (FIG. 2) has a hexagonal cross-sectional shape and is sized for receipt within the hexagonal socket 18 of bone screw 10 (FIG. 1).

Head 32 of driver 30 is illustrated in FIGS. 4A-4C. Head 32 includes first end 34 that is securable to handle 31 of driver 30 (FIG. 2) and second end 36 opposite first end 34 that is sized and shaped for receipt within socket 18 of bone screw 10 (FIG. 1). Longitudinal axis 38 of head 32 extends from first end 34 to second end 36 of head 32. Head 32 includes a plurality of sides 40 that cooperate to form head 32 having a hexagonal cross-sectional shape (in a direction perpendicular to longitudinal axis 38 of head 32). Borders 42 extend between adjacent sides 40. Head 32 may be constructed of metal or another suitable material. Like bone screw 10 (FIG. 1), head 32 may also be cannulated to accommodate a guide wire (not shown).

Referring to FIGS. 4A-4C, second end 36 of head 32 is offset relative to first end 34 of head 32. More particularly, second end 36 of head 32 is rotatably offset about longitudinal axis 38 relative to first end 34 of head 32. The degree to which second end 36 of head 32 is rotatably offset from first end 34 of head 32, which is labeled in FIG. 4C as angle α, may vary depending on the particular application. For example, angle α may be as small as 1 degree, 3 degrees, 5 degrees, or 7 degrees, and as large as 9 degrees, 11 degrees, 13 degrees, 15 degrees, or more. Angle α is formed between the radius from longitudinal axis 38 to border 42 at first end 34 of head 32 and the radius from longitudinal axis 38 to the same border 42 at second end 36 of head 32. In this embodiment, sides 40 of head 32 (and borders 42 between sides 40 of head 32) are helically disposed about longitudinal axis 38.

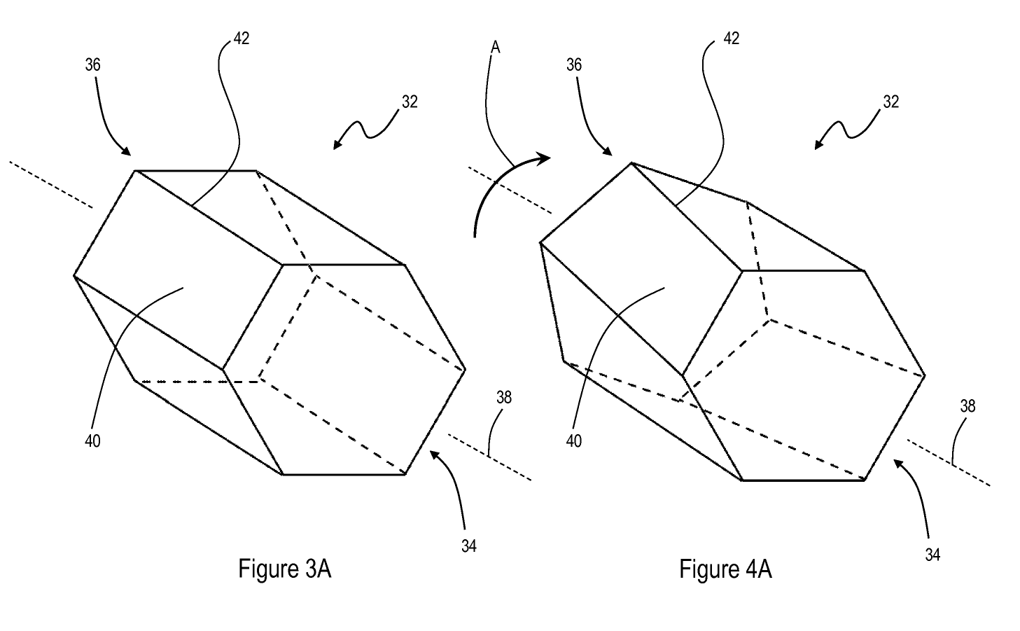

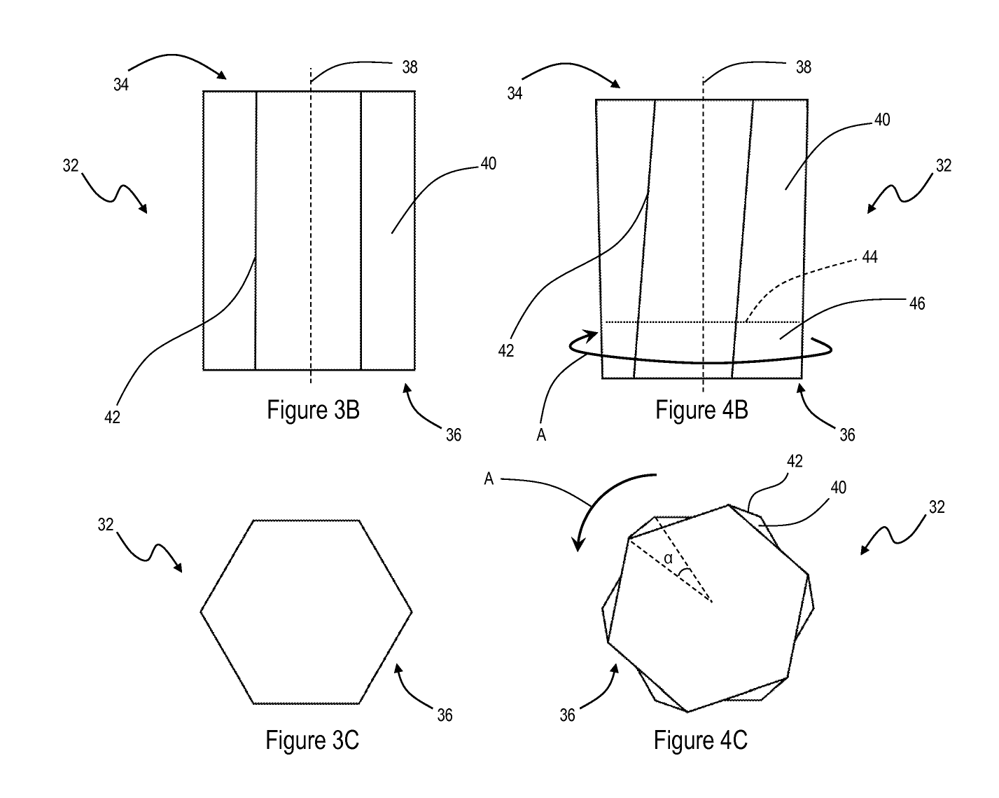

An exemplary method of manufacturing head 32 of driver 30 is described below with reference to FIGS. 3A-3C and 4A-4C.

First, as shown in FIG. 3A, head 32 may be machined or cast such that second end 36 of head 32 is substantially aligned with first end 34 of head 32 along longitudinal axis 38. In this embodiment, borders 42 between sides 40 of head 32 may extend substantially parallel to one another and to longitudinal axis 38. According to an exemplary embodiment of the present disclosure, head 32 may be in the shape of a right prism, with first and second ends 34, 36, extending in parallel and sides 40 extending at right angles relative to first and second ends 34, 36. According to another exemplary embodiment of the present disclosure, head 32 may be in the shape of a regular prism, with all sides 40 of head 32 being equal in size.

Next, as shown in FIGS. 4A-4C, second end 36 of head 32 is rotated about longitudinal axis 38 in the direction of arrow A relative to first end 34 of head 32. Although arrow A extends in a clockwise direction in the view of FIG. 4A, it is within the scope of the present disclosure than arrow A may extend in a counterclockwise direction, as described further below. This step may involve gripping or clamping both ends of head 32 and holding one end (e.g., first end 34) stationary while applying torque to the other end (e.g., second end 36). Head 32 should be twisted to such an extent that head 32 remains in the desired helical shape even after head 32 is no longer subject to torque.

Finally, one or both ends 34, 36, of head 32 may be trimmed to remove any regions that may have been damaged when clamping and twisting head 32. For example, as shown in FIG. 4B, head 32 may be trimmed along cut line 44 to remove region 46 from second end 36 of head 32 that may have become deformed under the clamping force and/or the torque.

Referring back to FIGS. 1 and 2, the surgeon is able to drive bone screw 10 into a patient's bone (not shown) using driver 30. First, the surgeon places second end 36 of head 32 into socket 18 of bone screw 10. Then, the surgeon rotates handle 31 of driver 30. As head 32 of driver 30 begins to rotate within socket 18 of bone screw 10, sides 40 and/or borders 42 of head 32 engage walls 16 surrounding socket 18 of bone screw 10 and transmit the rotational movement of driver 30 to bone screw 10. Rotating driver 30 in a clockwise direction R, as shown in FIG. 2, causes bone screw 10 to rotate in a clockwise direction and may drive bone screw 10 into a patient's bone. Rotating driver 30 in a counterclockwise direction, opposite arrow R, causes bone screw 10 to rotate in a counterclockwise direction and may separate bone screw 10 from the patient's bone.

Referring still to FIGS. 1 and 2, the helical shape of head 32 enables sides 40 and/or borders 42 of head 32 to lock against walls 16 surrounding socket 18 of bone screw 10. When using a standard driver head in the shape of a regular prism (such as head 32 of FIG. 3A), small gaps may exist between the standard head and socket 18 of bone screw 10 due to machining tolerances in forming a head that is small enough to fit within socket 18 and socket 18 that is large enough to receive the head. However, the helical shape of head 32 (FIG. 4A) tightens the fit between head 32 and walls 16 surrounding socket 18 of bone screw 10. For example, along second end 36, borders 42 of head 32 may be in point-contact with walls 16 surrounding socket 18 of bone screw 10. The locked engagement between head 32 and bone screw 10 enables the surgeon to position and orient bone screw 10 against the patient's bone by moving driver 30, rather than having to hold and manipulate bone screw 10 itself. Also, this locked engagement reduces the likelihood that bone screw 10 will separate from driver 30 when rotating driver 30. Angle α (FIG. 4C) may be selected to provide a desired amount of locking while still allowing head 32 to be inserted into and removed from socket 18 of bone screw 10 when necessary.

The twisting process described above may also increase the torsional strengthen of head 32 by leaving behind residual stresses in head 32. According to an exemplary embodiment of the present invention, and as shown in FIG. 2, head 32 may be pre-stressed in the same direction that head 32 will be rotated to implant bone screw 10. In other words, arrow A and arrow R may face in the same direction. When the surgeon rotates driver 30 in the clockwise direction R, head 32 will be pre-stressed to resist opposing forces from bone screw 10 (FIG. 1) in the direction of arrow F. It is within the scope of the present invention that another driver head may be provided that has been pre-stressed in the opposite direction of head 32 (opposite arrow A) for use when driver 30 must be rotated in the counter-clockwise direction (opposite arrow R), such as when removing bone screw 10 from a patient's bone (not shown). Alternatively, the same head 32 may be used, with head 32 being flipped such that second end 36 is secured to handle 31 of driver 30 instead of first end 34.

Known driver heads may be machined or cut into a helical shape to encourage locking between the head and the bone screw. However, due at least in part to the small size of such driver heads and the close machining tolerances required, such machining processes are more time consuming and expensive than the twisting process described above. Also, screw heads that are simply machined or cut into a helical shape lack the residual stresses described above.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of manufacturing an orthopedic tool comprising the steps of:
providing a head shaped in a first configuration, the head having a longitudinal axis, a first end, a second end, and a plurality of sides that extend from the first end to the second end, the plurality of sides defining a non-circular cross section in a direction perpendicular to the longitudinal axis;
applying torque to the head to shape the head into a second configuration that differs from the first configuration, the plurality of sides extending helically about the longitudinal axis in the second configuration; and
coupling the head to a handle.

2. The method of claim 1, wherein the applying torque step comprises rotating the second end relative to the first end of the head in a first direction that is the same as an intended direction of rotating the handle to drive a bone screw, whereby the bone screw applies a force to the head in a second direction opposite the first direction when rotating the handle in the intended direction.

3. The method of claim 1, wherein the head is in the shape of a regular prism in the first configuration.

4. The method of claim 1, wherein the second end of the head is aligned with the first end of the head along the longitudinal axis in the first configuration.

5. The method of claim 4, wherein the second end of the head is rotatably offset from the first end of the head about the longitudinal axis in the second configuration.

6. The method of claim 1, wherein the head remains shaped in the second configuration after the applying torque step.

7. The method of claim 1, further comprising the step of trimming at least one of the first and second ends of the head after the applying torque step.

8. A method of manufacturing an orthopedic tool for use with a bone screw, the bone screw defining a socket with a non-circular cross section, the method comprising the steps of:
providing a head shaped in a first configuration, the head having a longitudinal axis, a first end, a second end, and a plurality of sides that extend from the first end to the second end, the plurality of sides defining a non-circular cross section in a direction perpendicular to the longitudinal axis;
applying torque to the head to shape the head into a second configuration that differs from the first configuration, the head sized to be inserted and removed from the socket of the bone screw while shaped in the second configuration; and
coupling the head to a handle.

9. The method of claim 8, further comprising the steps of:
after the applying torque step, inserting the head of the orthopedic tool into the socket of the bone screw; and
after the inserting step, rotating the handle of the orthopedic tool to turn the bone screw, the bone screw applying a force to the head in a direction opposite the applying torque step.

10. The method of claim 8, wherein the head is in the shape of a regular prism in the first configuration.

11. The method of claim 8, wherein the second end of the head is aligned with the first end of the head along the longitudinal axis in the first configuration.

12. The method of claim 11, wherein the second end of the head is rotatably offset from the first end of the head about the longitudinal axis in the second configuration.

13. The method of claim 8, wherein the head remains shaped in the second configuration after the applying torque step.

14. The method of claim 8, further comprising the step of trimming at least one of the first and second ends of the head after the applying torque step.

15. An orthopedic tool for use with a bone screw, the bone screw defining a socket with a non-circular cross section, the orthopedic tool comprising:
a handle; and
a head shaped in a second configuration and coupled to the handle, the head having a longitudinal axis, a first end, a second end, and a plurality of sides that extend from the first end to the second end, the plurality of sides defining a non-circular cross section in a direction perpendicular to the longitudinal axis, the head manufactured by the steps of:
providing the head shaped in a first configuration that differs from the second configuration; and
applying torque to the head to shape the head into the second configuration, the head sized to be inserted and removed from the socket of the bone screw while shaped in the second configuration.

16. The orthopedic tool of claim 15, wherein the head is hexagonal in cross section.

17. The orthopedic tool of claim 15, wherein the second end of the head is aligned with the first end of the head along the longitudinal axis in the first configuration.

18. The orthopedic tool of claim 17, wherein the second end of the head is rotatably offset from the first end of the head about the longitudinal axis in the second configuration.

19. The orthopedic tool of claim 15, further comprising an additional head that is interchangeably coupled to the handle, the additional head having a longitudinal axis, a first end, a second end, and a plurality of sides that extend from the first end to the second end, the additional head shaped in a third configuration that differs from the first and second configurations.

20. The orthopedic tool of claim 19, wherein the additional head is manufactured by rotating the second end relative to the first end of the additional head in a direction opposite the applying torque step.
